(19) United States
(12) Patent Application Publication
(10) Pub. No.: US 2026/0013781 A1
(43) Pub. Date: Jan. 15, 2026

(54) TARGET RECOGNITION METHODS BASED ON ELECTROENCEPHALOGRAM SIGNALS IN NATURAL READING ENVIRONMENT

(71) Applicant: TIANJIN UNIVERSITY, Tianjin (CN)

(72) Inventors: Yanru BAI, Tianjin (CN); Qi TANG, Tianjin (CN); Guangjian NI, Tianjin (CN); Dong MING, Tianjin (CN); Ran ZHAO, Tianjin (CN); Hongxing LIU, Tianjin (CN); Jinghan YANG, Tianjin (CN); Shihong JIANG, Tianjin (CN)

(73) Assignee: TIANJIN UNIVERSITY, Tianjin (CN)

(21) Appl. No.: 19/334,750

(22) Filed: Sep. 19, 2025

Related U.S. Application Data
(63) Continuation-in-part of application No. 19/026,709, filed on Jan. 17, 2025.

(30) Foreign Application Priority Data
Feb. 18, 2024 (CN) 202410181631.1

(51) Int. Cl. A61B 5/378; A61B 5/00; G06N 3/0442; G06N 3/0464

(52) U.S. Cl. CPC A61B 5/378; A61B 5/0006; A61B 5/7203; A61B 5/7225; A61B 5/726; A61B 5/7267; A61B 5/742; G06N 3/0442; G06N 3/0464

(57) ABSTRACT

A target recognition method based on an electroencephalogram (EEG) signal in a natural reading environment is provided, including: presenting a fuzzy semantic target to a subject through a user interaction window on a display device; acquiring EEG signals of the subject via a wireless EEG acquisition device to obtain a target EEG signal corresponding to the fuzzy semantic target; determining a binary classification result corresponding to the fuzzy semantic target based on the target EEG signal through a trained EEG classification model; in response to the binary classification result indicating that the fuzzy semantic target is recognized, determining a semantic category to which the fuzzy semantic target belongs; and controlling, based on the semantic category, the display device to highlight annotations on the user interaction window; wherein highlighting annotations includes highlighting a text or an image related to the fuzzy semantic target and displaying associated information of the semantic category.

### CROSS REFERENCE TO RELATED APPLICATIONS

This application is a continuation-in-part of U.S. application Ser. No. 19/026,709, filed on Jan. 17, 2025, which claims priority to Chinese Patent Application No. 202410181631.1, filed on Feb. 18, 2024, the entire contents of which are incorporated herein by reference.

### TECHNICAL FIELD

The present disclosure relates to the field of cognitive science and artificial intelligence, and in particular, to target recognition methods based on electroencephalogram (EEG) signals in natural reading environment.

### BACKGROUND

Cognitive science is an advanced and cutting-edge discipline that studies human sensation, perception, mental states, brain thinking processes, and information processing. Research in this field is of great significance in uncovering the mysteries of the human brain. The classification of cognitive task patterns is widely used in the development of brain-machine interaction systems, the study of brain mechanisms, and the investigation of the pathogenesis of various brain diseases. Studies have shown that when subjects engage in a cognitive response related to a task, it involves complex cognitive processing stages, such as decision-making and working memory updating.

With the development of information technology, the importance of physiological signals has been discovered. Among them, EEG signals generated by the brain can reflect human brain activity, as well as information such as electrophysiological, and pathological. Specifically, when the brain is in an active state without specific external stimuli, the nervous system spontaneously generates rhythmic voltage changes, forming spontaneous EEG. Evoked EEG refers to the regular voltage changes in specific brain regions caused by external stimuli such as light, sound, electrical, or other types of task stimuli, which are known as event-related potentials (ERPs), also referred to as evoked potentials. Designing special experimental paradigms to conduct experiments, evoking EEG, and observing its variation process is an important method for brain cognition research. Analyzing EEG signals to study brain cognitive responses and exploring the relationship between semantic understanding and brain cognition has always been a key focus in EEG signal research.

Significant progress has been made in brain cognition research based on EEG signals. The main work includes studying the differences in working memory load and attention levels under different task conditions, the effects of emotions, cognition, and attention on ERPs, the decoding characteristics of specific brain regions on text content, the neural mechanisms of visual-spatial cognition under multitask conditions, and the assessment of neural damage in patients with mild cognitive impairment during task switching, etc. These studies involve both the impact of external stimuli on EEG and the analysis of brain neural mechanisms based on EEG signals.

In research on the brain's cognition of textual content, existing studies often adopt paradigms with clear objectives, usually focusing on specific texts. However, in natural reading environments, textual information may be semantically ambiguous, such as when the cognitive object refers to a category of things rather than a specific thing, which creates challenges for studying EEG signals in natural reading environments to analyze the brain's cognitive processes.

### SUMMARY

One or more embodiments of the present disclosure provide a target recognition method based on an electroencephalogram (EEG) signal in a natural reading environment executed by a processor, comprising: presenting a fuzzy semantic target to a subject through a user interaction window on a display device; acquiring EEG signals of the subject via a wireless EEG acquisition device to obtain a target EEG signal corresponding to the fuzzy semantic target; determining a binary classification result corresponding to the fuzzy semantic target based on the target EEG signal through a trained EEG classification model; in response to the binary classification result indicating that the fuzzy semantic target is recognized, determining a semantic category to which the fuzzy semantic target belongs; and controlling, based on the semantic category, the display device to highlight annotations on the user interaction window; wherein highlighting annotations includes highlighting a text or an image related to the fuzzy semantic target and displaying associated information of the semantic category in an auxiliary area of the user interaction window.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, where:

FIG. 1 is a flowchart illustrating a process of an exemplary target recognition method based on an EEG signal in a natural reading environment according to some embodiments of the present disclosure;

FIG. 2 is a flowchart illustrating an exemplary process of generating a negative feedback instruction and a positive feedback instruction according to some embodiments of the present disclosure;

FIG. 3 is a flowchart illustrating an exemplary process of generating an execution instruction according to some embodiments of the present disclosure;

FIG. 4 is a flowchart illustrating an exemplary process of training an EEG classification model according to some embodiments of the present disclosure;

FIG. 5 is an explanatory diagram of a stimulus material used according to some embodiments of the present disclosure;

FIG. 6 is a schematic diagram illustrating an exemplary experimental process according to some embodiments of the present disclosure;

FIG. 7 is a schematic diagram illustrating an exemplary structure of an EEG classification model according to some embodiments of the present disclosure;

FIG. 8 is a schematic diagram illustrating an exemplary Module A in an EEG classification model according to some embodiments of the present disclosure; and

FIG. 9 is a schematic diagram illustrating an exemplary Module B in an EEG classification model according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure.

It should be understood that the terms “system”, “device”, “unit” and/or “module” as used herein are a way to distinguish between different components, elements, parts, sections, or assemblies at different levels. However, the words may be replaced by other expressions if other words accomplish the same purpose.

As shown in the present disclosure and the claims, unless the context clearly suggests an exception, the words “a”, “an”, “one” and/or “the” do not refer specifically to the singular but may also include the plural. Generally, the terms “including” and “comprising” suggest only the inclusion of clearly identified steps and elements that do not constitute an exclusive list, and the method or apparatus may also include other steps or elements.

It should be appreciated that the preceding or following operations are not necessarily performed in an exact sequence. Instead, steps can be processed in reverse order or simultaneously. Also, it is possible to add other operations to these processes or remove a step or steps from them.

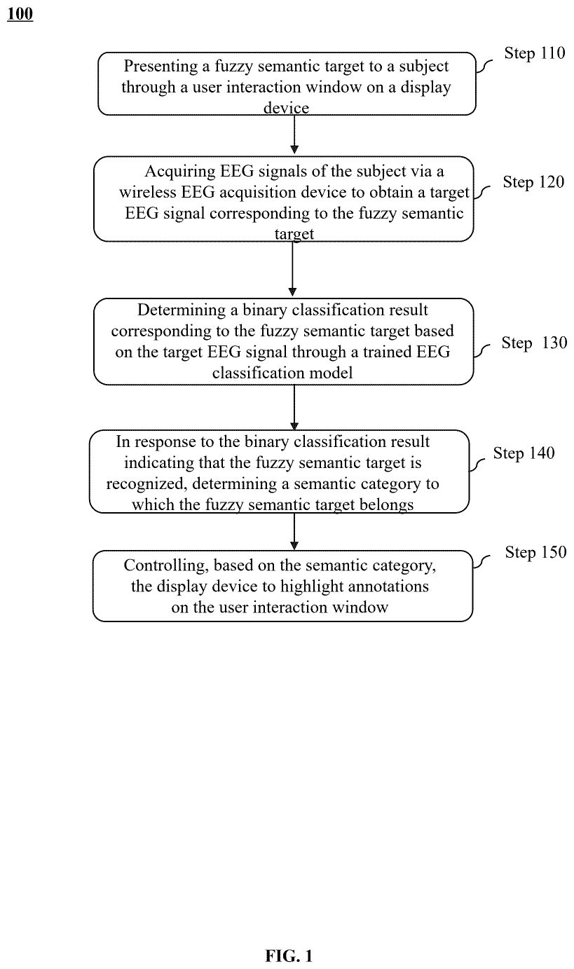

FIG. 1 is a flowchart illustrating a process of an exemplary target recognition method based on an EEG signal in a natural reading environment according to some embodiments of the present disclosure.

As shown in FIG. 1, a process 100 may include the following steps. In some embodiments, the process 100 may be executed by a processor.

The processor may process data and/or information related to the target recognition method based on the EEG signal in the natural reading environment. The processor may execute program instructions based on such data, information, and/or processing results to execute one or more of the functions described in the present disclosure. In some embodiments, the processor may include one or more sub-processing devices (e.g., a single-core processing device, a multi-core processing device, etc.). By way of example only, the processor may include a central processing unit (CPU), an application-specific integrated circuit (ASIC), etc., or any combination of the above.

In step 110: presenting a fuzzy semantic target to a subject through a user interaction window on a display device.

The display device refers to a device with display functions, such as an LCD display, a VR headset, etc.

The user interaction window refers to a window used to interact with the subject. The subject refers to an experimenter who is subjected to the EEG experiment, i.e., a user.

The fuzzy semantic target refers to a cognitive object with polysemy or category generalization, such as the text “apple” (which may refer to the fruit or the company) or the text “tiger” (which may refer to the animal or the brand). The fuzzy semantic target may include, but are not limited to, textual forms, image forms, etc.

In some embodiments, the processor may randomly select the fuzzy semantic target from a preset semantic library. To realize some coverage, the fuzzy semantic target includes four categories including a name of people, means of transportation, an animal, and a fruit. Each category is embodied as a plurality of fuzzy semantic targets, e.g., the category of “a name of people” includes two fuzzy semantic targets: “Zhang San” and “Li Si”. The preset semantic library may be manually constructed based on a large amount of data, including a large number of fuzzy semantic targets.

In step 120: acquiring EEG signals of the subject via a wireless EEG acquisition device to obtain a target EEG signal corresponding to the fuzzy semantic target.

The wireless EEG acquisition device refers to an EEG signal acquisition device, such as a wireless electrode cap, a wireless EEG machine, or the like.

The target EEG signal refers to an EEG signal recorded from the subject while reading the fuzzy semantic target.

In some embodiments, during the presentation of the fuzzy semantic target and immediately following period of blank frame, the processor may acquire the target EEG signal corresponding to the fuzzy semantic target via the wireless EEG acquisition device. For example, the presentation time of the ambiguous fuzzy target may be 2 seconds, the time of blank frame may be 0.2 seconds, and the EEG signals corresponding to the fuzzy semantic target may be signals acquired within 2.2 seconds.

In step 130: determining a binary classification result corresponding to the fuzzy semantic target based on the target EEG signal through a trained EEG classification model.

The EEG classification model may be a model configured to recognize and classify the fuzzy semantic target in the natural reading environment.

The binary classification result refers to a binary decision output of whether the fuzzy semantic target is recognized. For example, the binary classification result may include 0 and 1, where 1 indicates that the fuzzy semantic target is recognized and 0 indicates that the fuzzy semantic target is not recognized.

In some embodiments, the processor may input the target EEG signal corresponding to the fuzzy semantic target into the trained EEG classification model, and output the binary classification result corresponding to the fuzzy semantic target.

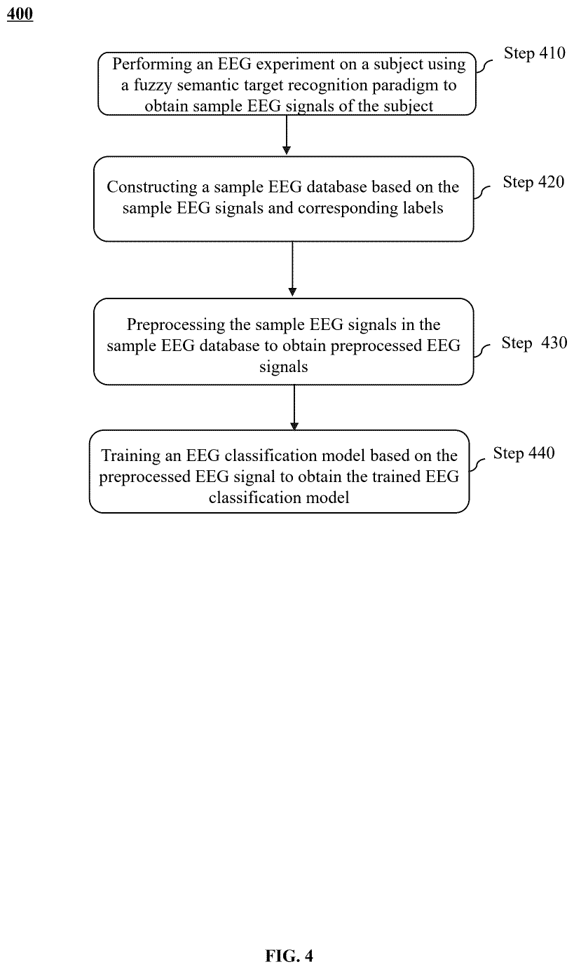

For more information on how to obtain the trained EEG classification model, please refer to the description of FIG. 4. For more information on how to determine the binary classification result corresponding to the fuzzy semantic target using the trained EEG classification model, please refer to the description of FIG. 4.

In step 140: in response to the binary classification result indicating that the fuzzy semantic target is recognized, determining a semantic category to which the fuzzy semantic target belongs.

The semantic category refers to a category divided according to different semantics. For example, the semantic categories to which fuzzy semantic targets belong may include four categories: name of people, means of transportation, animals, and fruits, as shown in Table 1 below.

TABLE 1
Categories of experimental materials and specific content thereof

| Cetegory | Specific Content | Content |
|---|---|---|
| Name of people | Zhang San, Li Si | 50*2 |
| Means of transportation | Train, Airplane, Ship, Bicyle, | 50*2 |
| | Bullet train | |
| Anminal | Parrot, Hedgehog, Crane, | 50*2 |
| | Steed, Kitten | |
| Fruit | Watermelon, Pineapple, | 50*2 |
| | Orange, Strawberyy, Grape | |

In some embodiments, in response to the binary classification result indicating that the fuzzy semantic target is recognized, the processor may query the semantic database to determine a first feature semantic that has the highest semantic similarity with the fuzzy semantic target, and determine a first feature category corresponding to the first feature semantic as the semantic category to which the fuzzy semantic target belongs.

The semantic database may be manually constructed based on a large amount of data and includes a plurality of first feature semantics and their corresponding first feature categories. The first feature semantic may be a historical fuzzy semantic target (including a historical text, a historical image, etc.), and the first feature category may be a semantic category corresponding to the first feature semantic, which may be manually annotated. When the fuzzy semantic target is in a text form, the semantic similarity between the fuzzy semantic target and the first feature semantic may be a text similarity; when the fuzzy semantic target is in an image form, the semantic similarity between the fuzzy semantic target and the first feature semantics may be an image similarity. The text similarity may be determined by a large language model, and the image similarity may be determined by an image recognition model.

In step 150: controlling, based on the semantic category, the display device to highlight annotations on the user interaction window.

In some embodiments, highlighting annotation includes highlighting a text or an image related to the fuzzy semantic target and displaying associated information of the semantic category in an auxiliary area of the user interaction window. The text or image related to the fuzzy semantic target refers to a text or an image that belongs to the same semantic category as the fuzzy semantic target.

The auxiliary area may be a sub-window, pop-up window, or similar element on the user interaction window.

The associated information of the semantic category includes texts, images, etc., that belong to the same semantic category as the fuzzy semantic target, as well as interaction options.

The interaction options refer to options for interacting with the subject, for example, “View Details”, “Play Video”, etc.

In some embodiments of the present disclosure, the category of the fuzzy semantic target is recognized in real time through EEG signals, automatically highlighting relevant texts/images in the user interaction window and displaying semantically associated information. This significantly reduces the cognitive load on users in understanding ambiguities, instantly converts neural decoding results into visual annotations, achieves seamless “brain-machine-environment” interaction, and transforms traditional static reading interfaces into dynamic interactive systems capable of perceiving users' cognitive focus in real time and proactively providing intelligent, multi-dimensional visual assistance. This effectively enhances information acquisition efficiency in natural reading scenarios.

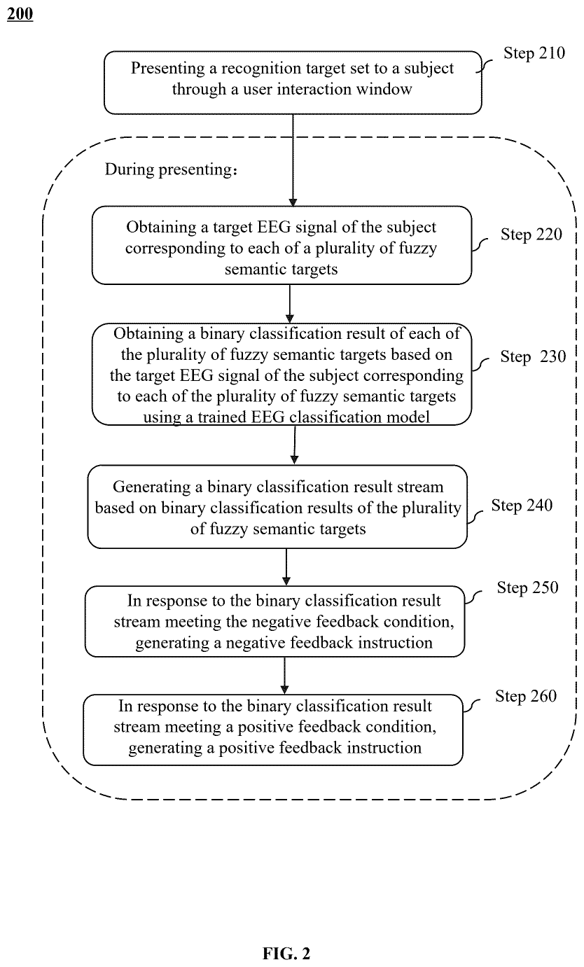

FIG. 2 is a flowchart illustrating an exemplary process of generating a negative feedback instruction and a positive feedback instruction according to some embodiments of the present disclosure.

As shown in FIG. 2, process 200 includes the following steps. In some embodiments, process 200 may be executed by a processor.

In step 210: presenting a recognition target set to a subject through a user interaction window.

The recognition target set refers to a collection of targets to be recognized by the subject. In some embodiments, the recognition target set includes a plurality of fuzzy semantic targets.

During the presentation process, the processor may execute steps 220 to 260.

In step 220: obtaining a target EEG signal of the subject corresponding to each of the plurality of fuzzy semantic targets.

In some embodiments, the processor may acquire the target EEG signal of the subject for each fuzzy semantic target based on a wireless EEG acquisition device. For related details, refer to step 120 in FIG. 1.

In step 230: obtaining a binary classification result of each of the plurality of fuzzy semantic targets based on the target EEG signal of the subject corresponding to each of the plurality of fuzzy semantic targets using a trained EEG classification model.

For related details, refer to the description of step 130 in FIG. 1.

In step 240: generating a binary classification result stream based on binary classification results of the plurality of fuzzy semantic targets.

The binary classification result stream refers to a sequence composed of binary classification results of the plurality of fuzzy semantic targets.

In some embodiments, the processor may arrange the plurality of corresponding binary classification results continuously output by the trained EEG classification model for the plurality of fuzzy semantic targets included in the recognition target set in the order of recognition, thereby forming the binary classification result stream. The binary classification result stream includes N consecutively generated binary classification results. The value of N may be preset manually.

In some embodiments, it is known that there are two scenarios: the binary classification result stream satisfies a negative feedback condition, and the binary classification result stream satisfies a positive feedback condition. For details, refer to steps 250 and 260 below.

In step 250: in response to the binary classification result stream meeting the negative feedback condition, generating a negative feedback instruction.

The negative feedback condition refers to a condition where the binary classification result stream is characterized as negative feedback. The negative feedback condition may be that a count of consecutive unrecognized instances in the binary classification result stream reaches a first frequency threshold. For example, the binary classification results of M consecutive recognitions are “unrecognized”.

The value of M of the first frequency threshold may be preset manually. In some embodiments, the value of M may also be positively correlated with an energy proportion of Theta band energy of the target EEG signal. The Theta band energy refers to signal energy within a frequency range of 4 Hz-8 Hz. The energy proportion of the Theta band energy refers to a ratio of the Theta band energy to the total energy of the target EEG signal. The Theta band energy may be obtained by the processor performing a Fast Fourier Transform (FFT) or wavelet transform on the target EEG signal in real time. The value of M is less than the value of N, which is the count of binary classification results included in the aforementioned binary classification result stream.

The negative feedback instruction refers to a control signal that reflects or alerts about negative feedback. In some embodiments, the negative feedback instruction may be configured to control a wearable wristband to produce a vibration or a microcurrent to alert the subject to attention deviation. A vibration intensity or a microcurrent intensity is positively correlated with an unrecognized count (also referred to as the count of unrecognized instances) in the binary classification result stream.

The unrecognized count refers to a count of binary classification results in the binary classification result stream that indicate the fuzzy semantic target is not recognized.

In some embodiments, in response to the binary classification result stream satisfying the negative feedback condition, the processor may automatically generate the negative feedback instruction according to a preset program.

In step 260: in response to the binary classification result stream meeting a positive feedback condition, generating a positive feedback instruction.

The positive feedback condition refers to a condition where the binary classification result stream is characterized as positive feedback (i.e., good recognition performance). The positive feedback condition may be that the count of consecutive recognized instances in the binary classification result stream reaches a second frequency threshold K. For example, the binary classification results of K consecutive recognitions are “recognized”.

The value K of the second frequency threshold may be preset manually. The value of K is less than the aforementioned value of N.

The positive feedback instruction refers to a control signal that reflects or alerts about positive feedback. In some embodiments, the positive feedback instruction may be configured to control a head-mounted playback device to play a positive prompt tone, thereby reinforcing the subject's focused cognitive state.

The positive prompt tone refers to an acoustic signal generated by the processor and output through the head-mounted playback device when the positive feedback condition is satisfied. The positive prompt tone may be preset manually, such as “ding-ding-ding”.

In some embodiments, in response to the binary classification result stream satisfying the positive feedback condition, the processor may automatically generate the positive feedback instruction according to a preset program.

In some embodiments of the present disclosure, through the continuous presentation of the recognition target set and the analysis of the binary classification result stream, a closed-loop system is created that performs real-time, automatic, and adaptive bidirectional physical regulation of the subject's cognitive state, converting abstract cognitive assessment into concrete physiological intervention. By linearly adjusting the intensity of tactile/auditory stimuli based on the count of unrecognized instances, the subject's optimal cognitive state can be maintained, significantly improving the training efficiency of fuzzy semantic target recognition.

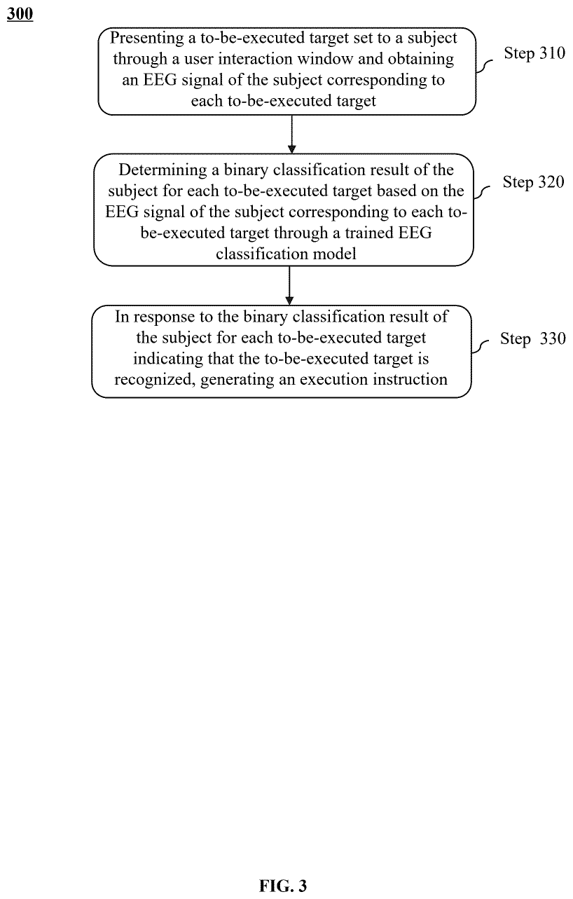

FIG. 3 is a flowchart illustrating an exemplary process of generating an execution instruction according to some embodiments of the present disclosure.

As shown in FIG. 3, process 300 includes the following steps. In some embodiments, process 300 may be executed by a processor.

In step 310: presenting a to-be-executed target set to a subject through a user interaction window and obtaining an EEG signal of the subject corresponding to each to-be-executed target.

The to-be-executed target set refers to a collection of a plurality of to-be-executed targets. The to-be-executed target refers to a specific action instruction that needs to be executed by a robotic arm, for example, “pick up the water cup”, “extend forward”, etc.

In some embodiments, the processor may randomly select to-be-executed targets from a preset rehabilitation action library to construct the to-be-executed target set.

In some embodiments, the processor may acquire the EEG signal of the subject for each to-be-executed target based on the wireless EEG acquisition device. For related details, refer to step 120 in FIG. 1.

In step 320: determining a binary classification result of the subject for each to-be-executed target based on the EEG signal of the subject corresponding to each to-be-executed target through the trained EEG classification model.

For an explanation of determining the binary classification result through the trained EEG classification model, refer to the related description in step 130 of FIG. 1.

In some embodiments, it is known that there are two scenarios: the binary classification result of the to-be-executed target indicates that the to-be-executed target is recognized, and the binary classification result of the to-be-executed target indicates that the to-be-executed target is not recognized. When the output result of the trained EEG classification model is “recognized” (i.e., the binary classification result indicates that the subject has produced a strong intention confirmation response to the to-be-executed target), it can be determined that the binary classification result of the to-be-executed target indicates that the to-be-executed target is recognized.

In step 330: in response to the binary classification result of the subject for each to-be-executed target indicating that the to-be-executed target is recognized, generating an execution instruction.

The execution instruction refers to a signal instruction that drives the rehabilitation robotic arm to move. In some embodiments, the execution instruction is configured to drive the rehabilitation robotic arm to execute the to-be-executed target with a preset trajectory and a preset posture.

The preset trajectory may be represented based on a three-dimensional spatial waypoint sequence (e.g., Cartesian coordinate path). The preset posture may be represented by a joint angle array of the rehabilitation robotic arm (e.g., [shoulder 30°, elbow 45°, wrist 0°]).

In some embodiments, in response to the binary classification result of the to-be-executed target indicating that the to-be-executed target is recognized, the processor may query an action database to identify a first feature action with the highest action similarity to the to-be-executed target, and determine a first trajectory and a first posture corresponding to the first feature action as the preset trajectory and preset posture for the to-be-executed target. The action similarity may be represented by a semantic similarity of the text corresponding to the to-be-executed target, and the semantic similarity may be confirmed based on a large language model.

The action database may be manually constructed based on a large amount of data and includes a plurality of first feature actions and their corresponding first trajectories and first postures. The first feature action may be a historical to-be-executed target. Among the historical trajectories and historical postures used in a plurality of historical executions corresponding to the historical to-be-executed target, the historical trajectory and historical posture of the historical execution with the best execution effect are taken as the first trajectory and first posture corresponding to the first feature action. The historical execution with the best execution effect refers to a historical execution where the to-be-executed target is not repeated after execution, i.e., the historical execution where the to-be-executed target is executed only once.

In some embodiments of the present disclosure, the binary classification result of EEG signals is cleverly applied to multi-instruction selection tasks, achieving a direct conversion from “intention confirmation” at the user's cognitive level to “complex robotic action execution” in the physical world. This provides individuals with mobility impairments with an efficient and safe device control manner that bypasses traditional motor pathways, constituting a significant functional improvement in assistive robot technology.

FIG. 4 is a flowchart illustrating an exemplary process of training an EEG classification model according to some embodiments of the present disclosure.

As shown in FIG. 4, process 400 includes the following steps. In some embodiments, process 400 may be executed by a processor.

In some embodiments, the trained EEG classification model is obtained through training based on a sample EEG database. Specifically, the following steps are included.

In step 410: performing an EEG experiment on a subject using a fuzzy semantic target recognition paradigm to obtain sample EEG signals of the subject.

The fuzzy semantic target recognition paradigm may be a paradigm used to study how well the subject recognizes fuzzy semantics.

In some embodiments, the fuzzy semantic target recognition paradigm includes a total of 8 blocks.

The block is an experiment block formed by dividing the stimulus material. An EEG experiment consists of a plurality of blocks, each block includes a plurality of trials, and the plurality of trials included in each block are run under a same condition in a preset order to minimize interference of random variability on the experiment.

The trial is a complete course of a single experimental manipulation or stimulus, including presentation of a stimulus material, response of a subject, and an acquired EEG signal. One trial corresponds to one stimulus material. Target trials are trials using a target stimulus material, and non-target trials are trials using a non-target stimulus material. In a single Block, a ratio of target trials to non-target trials may be 3:7, and an interval may exist between each trial.

The stimulus material may be a material used to elicit an EEG signal.

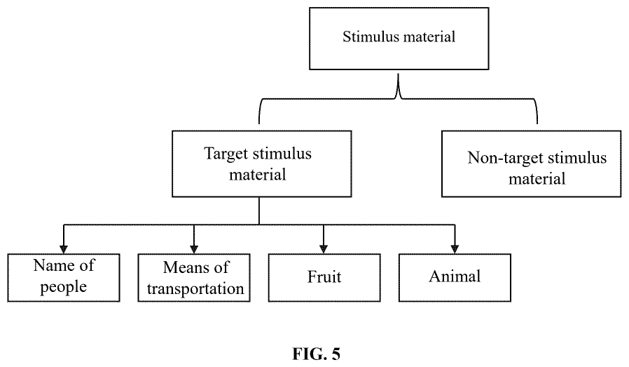

In some embodiments, the processor may select stimulus materials from network resources. The stimulus material is mainly sourced from online resources such as Baidu Baike, Renwu magazine, and WeChat public articles, and is homologous to a target work scenario. FIG. 5 is an explanatory diagram of a stimulus material used according to some embodiments of the present disclosure. As shown in FIG. 5, the stimulus materials include two types, namely a target stimulus material and a non-target stimulus material. The target semantic material is a stimulus material used to elicit a cognitive reflection from the subject on the fuzzy semantic target, including 4 semantic categories (names of people, means of transportation, fruits, and animals). The non-target semantic material is a stimulus material that avoids the individual preferences of the subject.

In order to form a contrast with the target stimulus material, the non-target stimulus material is selected from a more neutral news report, mainly from the media's reports related to the 20th National Congress of the Communist Party of China. A neutral material is chosen mainly to avoid interfering with an EEG feature of a subject due to its individual preferences.

In some embodiments, in the fuzzy semantic target recognition paradigm in the step 410, the stimulus material is a text of 15 to 20 words in length and is presented in a form of a news headline or a short sentence.

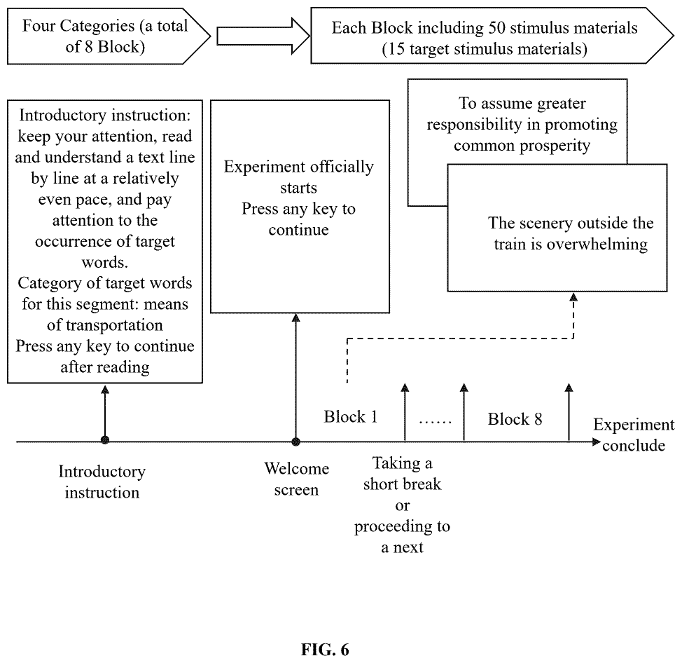

FIG. 6 is a schematic diagram illustrating an exemplary experimental process according to some embodiments of the present disclosure.

In some embodiments, as shown in FIG. 6, stimulus materials under each semantic category of the fuzzy semantic target are randomly composed into 2 Blocks, resulting in a total of 8 Blocks designed. A single Block contains 50 stimulus materials, including 15 target stimulus materials and 35 non-target stimulus materials (a ratio of 3:7). Each Block may complete 50 trials, and there exists a 0.2 s interval between each trial, i.e., for each subject, 50 EEG signals, each lasting 2.2 seconds (2 seconds for stimulus material presentation and 0.2 seconds for an interval), may be acquired as 50 sample EEG signals. In a single Block, the stimulus material appears pseudo-randomized, and it is guaranteed that a same category of fuzzy semantic target does not appear consecutively. In the entire Block of the experiment, all occurrences of stimulus materials are not repeated.

In some embodiments, the EEG experiment includes 2*count of semantic categories*50 trials. Each trial presents a stimulus material, and a total of 2*count of semantic categories*50 stimulus materials are presented, wherein the target stimulus material accounts for 30% and the non-target stimulus material accounts for 70%. For example, the entire EEG experiment consists of 50*8-400 trials, totaling 400 stimulus materials, among which 120 are target stimulus materials and 280 are non-target stimulus materials.

In some embodiments, for each block in the fuzzy semantic target recognition paradigm: the processor may present stimulus materials corresponding to the block to the subject in a predetermined sequence based on the fuzzy semantic object recognition paradigm, and control the wireless EEG acquisition device to acquire the sample EEG signals of the subject based on acquisition parameters; wherein the acquisition parameters include acquisition time periods and an acquisition frequency.

In some embodiments, the processor controls the wireless EEG acquisition device to acquire the sample EEG signals of the subject based on the acquisition parameters, including: controlling the wireless EEG acquisition device to perform a plurality of acquisitions on the subject at the acquisition frequency during different acquisition time periods; recording a sample EEG signal and a corresponding label when presenting each stimulus material and a sample EEG signal and a corresponding label during a period of blank frame following each stimulus material; after presentation of the stimulus materials for the block is completed, adjusting the acquisition parameters based on a quality of the sample EEG signals and a preset quality threshold; and controlling the wireless EEG acquisition device to reacquire the sample EEG signals of the subject corresponding to the stimulus materials of the block based on adjusted acquisition parameters.

As shown in FIG. 6, a subject begins an EEG experiment by first reading an introductory instruction. The instruction provides an overview of the experiment, including a requirement of the experiment and a semantic category (e.g., means of transportation) of the fuzzy semantic target for a current segment. The subject then enters a welcome screen, marking the official start of the experiment. Next, the subject progresses to an experimental Block 1, where stimulus materials (e.g., a target stimulus material such as “the scenery outside the train is overwhelming” and a non-target stimulus material such as “to assume greater responsibility in promoting common prosperity,” etc.) in the Block 1 are presented. After the stimulus materials in the Block 1 have been presented, the subject may take a short break before proceeding to a next Block. After all Blocks have been traversed, the experiment concludes.

In some embodiments, the experimental process of the EEG experiment may include step 2.1 to step 2.5.

In step 2.1: wearing a wireless EEG acquisition device for the subject and informing the subject of a semantic category of a fuzzy semantic target.

In step 2.2: randomly presenting, based on the fuzzy semantic target recognition paradigm, stimulus materials in a single Block in a preset sequence until all stimulus materials in the single Block have been traversed, and connecting a preceding stimulus material and a following stimulus material using a blank frame with a certain length.

In step 2.3: controlling the wireless EEG acquisition device to perform a plurality of acquisitions on the subject at the acquisition frequency during different acquisition time periods; and recording a sample EEG signal and a corresponding label when presenting each stimulus material and a sample EEG signal and a corresponding label during a period of blank frame following each stimulus material.

For example, a presentation time of each stimulus material may be 2 s, and a time of the blank frame may be 0.2 s, the sample EEG signal may be a 30-lead signal, and the corresponding label may be whether a fuzzy semantic target appears in the stimulus material. The corresponding label may be represented by 0 and 1, with 0 indicating that the fuzzy semantic target does not appear in the stimulus material and 1 indicating that the fuzzy semantic target appears in the stimulus material. The label may be marked manually.

The acquisition frequency refers to a frequency at which the wireless EEG acquisition device acquires EEG signals. The acquisition period refers to a period during which the wireless EEG acquisition device acquires EEG signals. The acquisition frequency and the acquisition period may be manually preset.

In step 2.4, after presentation of the stimulus materials for the block is completed, adjusting the acquisition parameters based on a quality of the sample EEG signals and a preset quality threshold.

The quality of a signal may be a parameter that assesses how good or bad the quality of the EEG signal is. The quality of a signal may be expressed by a numerical value, and the larger the value, the better the quality of the EEG signal.

In some embodiments, the processor may assess the quality of the sample EEG signal based on a user-type feature of the subject, an acquisition time feature of the sample EEG signal, and the sample EEG signal.

The user-type feature may include gender, age, education level, or the like.

The acquisition time feature may be a feature related to an acquisition time of the EEG signal. For example, the acquisition time feature may be categorized as morning, afternoon, evening, or alternatively, within 4 hours, between 4 and 10 hours, and more than 10 hours since the subject's last break. Under different acquisition time features, the states of the subject are different, which has different effects on the acquisition of the EEG signal.

In some embodiments, the processor may construct a plurality of first reference vectors based on historical user-type features, historical acquisition time features, and historical EEG signals corresponding to a plurality of historical subjects in a plurality of historical EEG experiments; construct a first target vector based on a user-type feature, an acquisition time feature, and an sample EEG signal of a current subject; determine a first reference vector that has a greatest vectorial similarity with the first target vector, compare a historical EEG signal included in the first reference vector with an EEG signal of the historical EEG signal after a historical actual preprocessing to determine a signal difference (represented by a difference in data amount) between the historical EEG signal before and after the preprocessing, calculate the reciprocal of a product of the signal difference and a preprocessing intensity, and designate the reciprocal as the quality of a corresponding signal. The preprocessing intensity is positively correlated with a count of filtrations in the preprocessing. For the preprocessing process, please refer to the description below.

In some embodiments of the present disclosure, assessing the quality of the EEG signal based on the user-type feature, the acquisition time feature, and the EEG signal is conducive to reducing a random error, by fully considering individual differences in EEG signals among different subjects and personal features of the subjects, improving the accuracy of assessing the quality of the EEG signal.

In some embodiments, the processor may also assess the quality of the sample EEG signal based on the user-type feature, the acquisition time feature, and the sample EEG signal using a quality assessment model.

The quality assessment model may be a model used to assess the quality of an EEG signal. In some embodiments, the quality assessment model may be a machine learning model, e.g., a Convolutional Neural Network (CNN). An input of the quality assessment model may include the user-type feature, the acquisition time feature, and the sample EEG signal, and an output of the quality assessment model may be the quality of the sample EEG signal.

In some embodiments, the quality assessment model may be obtained by training based on a plurality of sets of first training samples with a first training label.

In some embodiments, the first training sample may be obtained based on historical data. For example, a first reference vector may serve as a first training sample. The first training label may be historical signal quality corresponding to the first training sample, which may be represented by the reciprocal of a product of a historical signal difference and a historical preprocessing intensity.

In some embodiments, the first training sample is input into an initial quality assessment model, a first loss function is constructed based on the signal quality output from the initial quality assessment model and the first training label, and the initial quality assessment model is updated based on the first loss function, and when a first preset condition is satisfied, the training of the initial quality assessment model is completed, then a trained quality assessment model is obtained. The first preset condition may be that the first loss function converges, a count of iterations reaches a threshold, or the like.

In some embodiments of the present disclosure, the quality of a signal can be quickly and accurately determined using the quality assessment model, which is conducive to reducing computational resources occupied by obtaining a preprocessed EEG signal, thus reducing a time cost.

In some embodiments, the input of the quality assessment model may also include a stimulus material corresponding to the sample EEG signal.

In some embodiments, the first training sample further includes a historical stimulus material. In some embodiments, the processor may train the quality assessment model based on the first training sample that includes the historical stimulus material.

In some embodiments of the present disclosure, using the stimulus material as the input of the quality assessment model can be effective in targeting the assessment of the quality of the EEG signal in a natural reading environment, and thus improve the accuracy of the assessment result of the quality assessment model.

An ambient light intensity feature refers to a light feature of a surrounding environment in which the EEG experiment is performed, and the ambient light intensity feature may include a light brightness, a light color system (warm, cool, etc.), and so on.

A material carrier feature refers to a presentation carrier of the stimulus material. For example, if the stimulus material is a text, the material carrier feature may be a paper text or an electronic text, etc.

In some embodiments, the input of the quality assessment model may also include the ambient light intensity feature and the material carrier feature.

In some embodiments, the first training sample further includes a historical ambient light intensity feature and a historical material carrier feature corresponding to the historical EEG signal. The processor may train the quality assessment model based on the first training sample including the historical ambient light intensity feature and the historical material carrier feature.

In some embodiments of the present disclosure, considering the effects of different environments on the subject can be effective in targeting the assessment of the quality of the EEG signal in the natural reading environment, and thus improve the assessment effect of the quality assessment model.

In some embodiments, the quality of a signal is also configured to determine whether to reject the EEG signal. If the quality of a signal is lower than the preset quality threshold, a sample EEG signal corresponding to the signal may be rejected. The preset quality threshold may be predetermined by a technician based on experience.

In some embodiments, after presentation of the stimulus materials for the block is completed, in response to the quality of the sample EEG signal being less than the preset quality threshold, the processor may adjust the acquisition parameters to resample.

The acquisition parameters may be adjusted by increasing the acquisition frequency by a preset adjustment amount, which may be manually preset.

In some embodiments, the processor may also generate a plurality of candidate acquisition parameters, determine a rejection probability of each of the plurality of candidate acquisition parameters using a parameter determination model, the parameter determination model being a machine learning model, and determine the acquisition parameter based on the rejection probability.

In some embodiments, for each subject that needs to be re-take the EEG experiment, the processor may randomly combine ambient light intensity features, material carrier features, and acquisition period features corresponding to a plurality rejected EEG signals to determine a plurality of sets of candidate acquisition parameters for each subject.

In some embodiments, the processor may also randomly combine ambient light intensity features, material carrier features, and acquisition period features corresponding to a plurality of rejected EEG signals of a plurality of subjects, and randomly match with a plurality of subjects that need to re-take the EEG experiment to determine a plurality of candidate acquisition parameters corresponding to the plurality of subjects.

The rejection probability refers to a probability that EEG signal is rejected when the EEG experiment is conducted according to the candidate acquisition parameter. The rejection probability may be a Boolean value in a range of 0 to 1, wherein 1 indicates being rejected and 0 indicates being not rejected.

The parameter determination model may be a model that determines the acquisition parameter. The parameter determination model may be a machine learning model. For example, the parameter determination model may be a CNN, etc.

In some embodiments, an input of the parameter determination model may include the candidate acquisition parameter, the stimulus material, an acquisition device parameter, and an output of the parameter determination model may be the rejection probability of the candidate acquisition parameter.

In some embodiments, the parameter determination model may be obtained by training based on a plurality of sets of second training samples with a second training label.

In some embodiments, a second training sample may be composed of a historical acquisition parameter, a historical stimulus material, and a historical acquisition device parameter. The second training label may be whether the historical EEG signal corresponding to the second training sample is rejected. The processor may determine whether a subsequently acquired historical EEG signal corresponding to the second training sample is rejected in accordance with step 3.3 and step 3.4, and the second training label is manually labeled.

The training of the parameter determination model may refer to the above training process of the quality assessment model, which will not be repeated here.

In some embodiments, the processor may randomly select one of one or more non-rejected candidate acquisition parameters as the acquisition parameter.

In some embodiments of the present disclosure, performing resampling based on the rejection probability of the EEG signals is conducive to fully considering different fluctuations of EEG signals of different subjects during different acquisition periods.

In step 2.5, controlling the wireless EEG acquisition device to reacquire the sample EEG signals of the subject corresponding to the stimulus materials of the block based on adjusted acquisition parameters.

In some embodiments, the acquisition parameters also include an ambient light intensity feature and a stimulus material carrier feature. Based on the adjusted acquisition parameters, the wireless EEG acquisition device is controlled to re-acquire the sample EEG signals of the subject corresponding to the stimulus materials of the block, including: controlling the wireless EEG acquisition device to perform a plurality of acquisitions on the subject at the acquisition frequency during the different acquisition periods, under different ambient light intensity features, and under different material carrier features.

In some embodiments of the present disclosure, considering the ambient light intensity feature and the material carrier feature is conducive to simulating the natural reading environment and improving the quality of data in the EEG database.

In some embodiments, after a short break, repeating the step 2.2 and the step 2.5, the stimulus material in the next Block is represented until all Blocks have been traversed. A length of the break may be set by default or by a processor or by a technician depending on a situation of the subject.

In some embodiments, for a subject whose count of rejections exceeds a count threshold, the processor may re-conduct an experiment on the subject in accordance with acquisition combination parameters. The acquisition combination parameters may include a combination of a subject and an acquisition time feature.

The count of rejections refers to a total count of rejection times of EEG signals corresponding to a plurality of trials in a single EEG experiment of a subject, i.e., a count of rejected sample EEG signals under the same acquisition parameters. For example, if 50 signal acquisitions are performed under certain acquisition parameters and 12 of them are rejected, the count of rejections is 12. The larger the count of rejections, the more trials (sample EEG signals corresponding to the trials) are rejected in the EEG experiment. The count threshold may be preset manually.

In some embodiments, the processor may obtain a count of rejections of each subject in a respective EEG experiment. For a subject whose count of rejections exceeds the count threshold, the processor may re-obtain the sample EEG signal by re-conducting the EEG experiment on the subject based on an acquisition period feature.

In step 420: constructing a sample EEG database based on the sample EEG signals and corresponding labels.

In some embodiments, step 420 may specifically include the following steps 3.1 to 3.5.

In step 3.1: calculating a power spectrum of each lead signal.

Lead refers to an electrode configuration manner, and different leads may record EEG signals in different regions of the brain. The lead signal refers to an EEG signal captured and recorded through the lead, i.e., the lead signal is a manifestation of the EEG signal.

The power spectrum of the lead signal refers to a plot of a power distribution of the lead signal in a frequency domain. In a power spectrum plot, an X-axis represents a signal frequency, and a Y-axis represents a power density corresponding to a frequency.

In some embodiments, the processor may calculate the power spectrum of each lead signal using a frequency-domain analysis manner. The frequency-domain analysis manner may include Fourier transform, or the like.

In step 3.2: labeling a lead signal with a value greater than twice a standard deviation of an average power spectral energy as a bad lead signal and supplementing using neighborhood interpolation.

The bad lead signal refers to a lead signal with a low-quality after being assessed.

In some embodiments, the processor may calculate a power spectrum energy of each lead signal based on the power spectral plot, calculate an average and standard deviation of power spectrum energies of a plurality of lead signals, and label the lead signal with a value greater than twice the standard deviation of the average power spectral energy as the bad lead signal and remove the bad lead signal, and supplement remaining lead signals using neighborhood interpolation, i.e., data of a bad lead signal is replaced by an average of neighboring lead signals.

In step 3.3: calculating, in a single trial, a median of a variance of lead signals, and a median of a difference between each lead signal and an average of the lead signals.

In step 3.4: labeling a trial in which either of the two medians is greater than twice a standard deviation and rejecting a sample EEG signal corresponding to the trial.

In step 3.5: corresponding retained sample EEG signals with labels corresponding to the retained sample EEG signals, and constructing the sample EEG database.

An EEG signal (30-lead signals) during a presentation time (2 s) of each stimulus material and a period (0.2 s) of blank frame following the stimulus material may be one sample. Whether the fuzzy semantic target appears in the stimulus material may be a sample label, with 0 indicating the fuzzy semantic target does not appear and 1 indicating the fuzzy semantic target appears.

In some embodiments of the present disclosure, by screening in terms of both the lead signal and the trial, removing data of the bad lead signal and rejecting the sample EEG signal corresponding to a portion of trials, and constructing the sample EEG database based on the screened sample EEG signal and labels corresponding to the screened sample EEG signal are conducive to providing high-quality data resources for the sample EEG database, thereby improving the recognition effect.

In step 430: preprocessing the sample EEG signals in the sample EEG database to obtain preprocessed EEG signals.

In some embodiments, the preprocessing includes filtering, downsampling, and denoising.

In some embodiments, a specific frequency of the EEG signal may be selected through filtering. For example, an unwanted frequency in the EEG signal is removed, and an interested frequency band of the EEG signal is retained. Filtering may be accomplished by, for example, a filter.

Downsampling refers to a process of reducing a sampling rate of the EEG signal to reduce an amount of data. Downsampling may be achieved through a downsampling function, etc.

Denoising refers to the reduction of noise interference in the EEG signal. Sources of noise may include eye movements, muscle movements, and so on. Denoising may be achieved by wavelet transform, independent component analysis (ICA), etc.

In some embodiments of the present disclosure, filtering, downsampling, and denoising the sample EEG signal is conducive to suppressing the environmental interference, reducing the amount of data and a processing time, and improving a signal-to-noise ratio of the EEG signal, thereby improving the accuracy of feature extraction and analysis.

In some embodiments, preprocessing may further include removing bioelectrical artifacts and residual noise from a downsampled EEG signal, wherein different acquisition parameters correspond to different discrimination thresholds, and the discrimination thresholds are related to a count of rejections of the sample EEG signals.

The downsampled EEG signal refers to an EEG signal that has been downsampled.

The residual noise refers to noise remaining in the downsampled EEG signal, for example, residual 50 Hz power frequency interference. The bioelectrical artifacts refer to non-EEG electrical activities caused by biological interference, for example, eye movement artifacts and myoelectric artifacts.

The discrimination threshold is a critical threshold for determining whether a signal component is noise or bioelectric artifact. In some embodiments, the processor may decompose the downsampled EEG signal into a plurality of components and determine the remaining noise and the bioelectric artifacts based on the discrimination threshold. For example, the discrimination threshold is a kurtosis threshold set to 5. Components in the downsampled EEG signal with a kurtosis greater than 5 are considered noise components.

In some embodiments, the discrimination threshold is negatively correlated with the count of rejections of the sample EEG signal.

In some embodiments of the present disclosure, by removing bioelectric artifacts and residual noise from the downsampled EEG signals, the data quality can be effectively improved, the discrimination threshold can be adaptively lowered based on the count of rejections associated with the acquisition parameters, the recognition sensitivity of bioelectric artifacts and residual noise can be significantly improved, and the false rejection rate of effective EEG components can be effectively reduced, which is conducive to improving the accuracy of subsequent processing.

In some embodiments, the pre-processing process may specifically include the following steps 4.1 to 4.4.

In step 4.1: obtaining a re-referenced EEG signal by re-referencing the EEG signals using an average of all lead signals as a reference datum.

The average of all lead signals refers to an average of a plurality of lead signals in a same trial.

A re-referenced EEG signal is a sample EEG signal that has been re-referenced. Re-referencing is a signal conversion process that uses the average of all lead signals as a reference to eliminate single-point reference deviations.

In step 4.2: obtaining a filtered EEG signal by removing, from the re-referenced EEG signal, noise below 0.5 Hz and above 80 Hz and power-line interference at 50 Hz using a band-pass filter and a notch filter.

The filtered EEG signal refers to a sample EEG signal after filtering.

The bandpass filter may be a Chebyshev II filter or the like; the notch filter may be an adaptive comb filter or the like, which are not limited here.

In step 4.3: obtaining a downsampled EEG signal by downsampling the filtered EEG signal according to a sampling theorem.

The sampling theorem may be Nyquist theorem or the like, and is not limited here.

In step 4.4: decomposing the downsampled EEG signal into a plurality of independent components using the ICA, calculating a frequency feature of each component; and removing bioelectrical artifacts and residual noise.

ICA refers to the independent component analysis algorithm, for example, maximum likelihood estimation (MLE), information maximization, or the like.

In some embodiments, the processor may utilize the ICA to decompose the downsampled EEG signal into a plurality of components that are independent of each other, e.g., a component that represents myoelectricity, a component that represents ocular electricity, etc., and set a discrimination threshold to remove each independent component in accordance with a probability, so as to remove the bioelectric artifacts and the residual noise.

In some embodiments of the present disclosure, the EEG signal is re-referenced based on the average of all lead signals, and then denoised using the band-pass filter and the notch filter; and downsampled and decomposed using the ICA to remove the bioelectrical artifacts and the residual noise, which is beneficial to reduce the periodic noise and improve the accuracy of recognizing and removing the bioelectric artifacts, thereby improving the quality of the EEG signal.

In step 440: training the EEG classification model based on the preprocessed EEG signal to obtain the trained EEG classification model.

In some embodiments, the processor may use the preprocessed EEG signal as a sample for training the EEG classification model, and the label corresponding to the sample is the binary classification result corresponding to the preprocessed EEG signal, that is, whether a fuzzy semantic target appears in the stimulus material.

In some embodiments, the processor may input the concatenated 120-dimensional integrated feature vector into a fully connected layer, iteratively updating the network weights until convergence with the goal of minimizing the cross-entropy loss function, to obtain the trained EEG classification model. The specific training process can be found in the following description.

In some embodiments, the processor may perform feature extraction on the preprocessed EEG signal.

In some embodiments, feature extraction may specifically include the following steps 5.1 to 5.4.

In step 5.1: obtaining a signal time-frequency plot of each lead by performing time-frequency analysis on the preprocessed EEG signals using continuous wavelet transform (CWT).

The signal time-frequency plot is a graph that shows the properties of the EEG signal in time and frequency. A horizontal coordinate of the signal time-frequency plot may denote time and a vertical coordinate of the signal time-frequency plot may denote frequency.

In step 5.2: obtaining a time-frequency feature set of an EEG signal of each lead by extracting an image feature of the signal time-frequency plot using a convolutional neural network.

The time-frequency feature set is a set of image features of the signal time-frequency plot extracted using a convolutional neural network. The image features may include texture features, edge features, etc. The time-frequency feature set characterizes joint distribution features of the sample EEG signals in both time and frequency dimensions, encompassing energy variations and frequency component distributions at different time points and across different frequency bands.

Variations in EEG signals across different time points and frequency bands can reflect features of brain activity. The time-frequency feature set facilitates the capture of dynamic changing information of EEG signals in both temporal and spectral dimensions, which is crucial for distinguishing EEG responses elicited by different fuzzy semantic targets. For example, textual stimuli of distinct semantic categories may evoke divergent EEG activities at specific time points and frequency ranges. Through the extraction and analysis of image features from signal time-frequency maps, fuzzy semantic targets can be identified with enhanced precision.

In step 5.3: obtaining a sample entropy feature vector of each trial by calculating a sample entropy feature of a preprocessed signal of each lead in the trial.

The sample entropy feature refers to an eigenvalue of a sample entropy of a preprocessed EEG signal, which characterizes the complexity and irregularity of the EEG signal in the time series. The smaller the sample entropy, the stronger the regularity of brain activity. In some embodiments, the processor may extract the sample entropy feature through a sample entropy calculation formula.

The sample entropy feature vector may be a vector constructed from sample entropy features of a plurality of preprocessed EEG signals of leads in a trial.

The sample entropy is a metric that measures the complexity and irregularity of time series. The sample entropy feature vector of EEG signals can reflect the complexity and regularity of brain activity. Different cognitive tasks cause the brain to generate EEG signals with varying degrees of complexity. The sample entropy feature vector quantifies differences in EEG signals with different degrees of complexity, providing the model with unique information about the brain's cognitive state, thereby helping the model better distinguish between EEG signals corresponding to fuzzy semantic targets and non-fuzzy semantic targets.

In step 5.4: obtaining a spatial feature vector for each trial by extracting a spatial feature of the preprocessed signal of each lead in the trial using a common spatial pattern.

The spatial feature may be used to characterize a spatial distribution of the preprocessed EEG signal across different leads. The processor may determine the spatial feature by computing a covariance matrix of the processed EEG signals for each trial.

The spatial feature vector may be a vector constructed from spatial features of the plurality of preprocessed EEG signals of leads in a trial, which represents the distribution differences and features of EEG signals at different locations (spaces) on the scalp. A spatial feature vector of 1*lead count (e.g., 1*30) may be obtained for each sample.

Different areas of the brain may have different EEG activity distribution patterns when processing different semantic information. The spatial feature vector may capture these spatial distribution features, provide information support in the spatial dimension for the model to identify fuzzy semantic targets, and enhance the model's recognition ability.

In some embodiments of the present disclosure, extracting the time-frequency feature set of the EEG signal of each lead based on the signal time-frequency plot of each lead, further obtaining the sample entropy feature vector, and further determining the spatial feature vector is conducive to obtaining dynamic changes of the EEG signal at different time and frequency and enhancing a nonlinear feature of the EEG signal.

In some embodiments, the processor may perform an EEG experiment on the subject using a fuzzy semantic target recognition paradigm to obtain sample EEG signals of the subject; construct the sample EEG database based on the sample EEG signals and corresponding labels; randomly divide the sample EEG signals in the sample EEG database into a training set and a test set, use the sample EEG signals as an input and the corresponding labels as a target output; train an EEG classification model using the sample EEG signals and the corresponding labels in the training set to obtain the trained EEG classification model which outputs the binary classification result; and apply the test set to the trained EEG classification model, and analyze performance and robustness of the trained EEG classification model to explore an association between an EEG activity and a fuzzy semantic target recognition activity.

For more information about performing EEG experiments on the subject based on the fuzzy semantic target recognition paradigm to obtain sample EEG signals of the subject and constructing the sample EEG database based on the sample EEG signals and corresponding labels, please refer to the relevant description of FIG. 4.

In some embodiments, the processor may randomly divide sample EEG signals in the sample EEG database into a training set and a test set, using the sample EEG signals as an input and a corresponding label as a target output. The label corresponding to the sample EEG signals is whether the fuzzy semantic target appears in the stimulus material corresponding to the sample EEG signals.

The training set may be a dataset configured to train internal parameters of a model. The test set may be a dataset configured to test a generalization ability of the model. After adjusting the internal parameters of the model using the training set, the test set may be used to determine whether the model is running properly and how well the model performs.

In some embodiments, applying the trained EEG classification model to the test set, and analyzing performance and robustness of the trained EEG classification model based on a classification result of the test set to explore an association between an EEG activity and a fuzzy semantic target recognition activity.

In some embodiments of the present disclosure, training the EEG classification model using training set and the test set helps improve the binary classification accuracy of the EEG classification model, and enhances its generalization capability and robustness.

In some embodiments of the present disclosure, by determining the fuzzy semantic target recognition paradigm, conducting the EEG experiment and acquiring the EEG signal, constructing the EEG database based on high-quality EEG signals, extracting the feature of the EEG database, and constructing the EEG classification model, the accuracy of the binary classification is enhanced. When a subject is cognitively processing a certain type of text, an EEG signal is recorded from the surface of the skull, and a cognitive process of the brain is parsed by studying the EEG signal, so as to provide experimental bases for cognitive disorders and other diseases.

In some embodiments, the processor may also divide the training set or test set based on a count of rejections.

The processor may divide a plurality counts of rejections corresponding to a plurality of EEG experiments into a plurality of levels according to a gradient (e.g., a count of rejections within 10 times is classified as a level A, a count of rejections between 10 and 50 times is classified as a level B, etc.), and sample EEG signals corresponding to the plurality of levels are divided into a plurality of groups, and samples may be randomly extracted from sample EEG signals in the plurality of groups in accordance with a preset ratio into the test set and the training set. The preset ratio may be set by default by the processor or preset manually.

In some embodiments, the processor may evaluate a valid value of the EEG classification model based on an output of the EEG classification model.

The valid value may be a parameter that measures the performance of the EEG classification model. The valid value may be expressed as a numerical value, and the larger the valid value, the better the performance of the EEG classification model.

In some embodiments, the processor may determine a ratio of a count of concordant and a total count of labels, multiplied by an evaluation factor, as the valid value. The count of concordant may be a count of tests in which the output of the model is consistent with the label across a plurality of tests of the model.

In some embodiments, evaluation factors for different test sets are different, and the evaluation factor for the test set correlates to the count of rejections. The higher the count of rejections corresponding to EEG signal samples in a test set, the smaller the evaluation factor corresponding to the test set.

In some embodiments of the present disclosure, determining the evaluation factor based on the count of rejections enables accurate judging of the EEG classification model even when the sample quality is poor.

In some embodiments of the present disclosure, classifying EEG signals first and then extracting the EEG signals based on a discrimination threshold and the count of rejections is helpful to safeguard a consistent distribution of levels of count of rejections in the training set and the test set, which effectively improves the training quality of the EEG classification model. In some embodiments, the trained EEG classification model includes an EEGNet module, a CNN-LSTM module, a time feature module, a spatial feature module, and an integration module. The processor may determine an abstract spatio-temporal feature vector based on preprocessed EEG signals using the EEGNet module, wherein the EEGNet module is a convolutional neural network; determine a dynamic time-frequency feature vector based on a time-frequency feature set using the CNN-LSTM module; determine a normalized signal complexity feature vector based on a sample entropy feature vector using the temporal feature module; determine a normalized spatial discriminative feature vector based on a spatial feature vector using the spatial feature module; concatenate the abstract spatio-temporal feature vector, the dynamic time-frequency feature vector, the normalized signal complexity feature vector, and the normalized spatial discriminative feature vector using the integration module to obtain an integrated feature vector; and obtain the binary classification result corresponding to the target EEG signal based on the integrated feature vector using the integration module, wherein the integration module is a feedforward neural network (FNN).

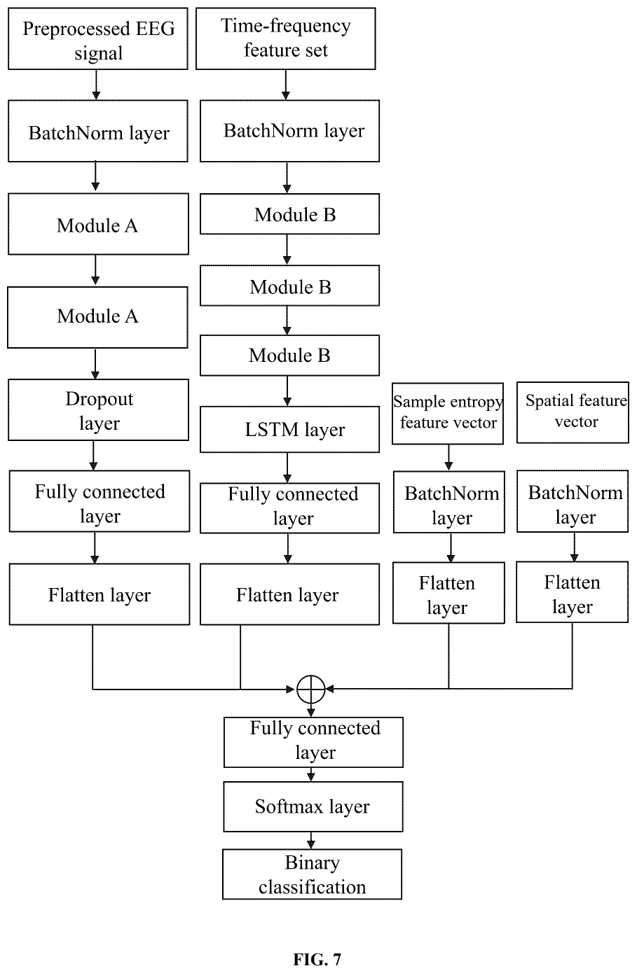

FIG. 7 is a schematic diagram illustrating an exemplary structure of an EEG classification model according to some embodiments of the present disclosure. As shown in FIG. 7, the EEG classification model includes following modules.

The EEGNet module takes preprocessed EEG signals as input and obtains an abstract spatio-temporal feature vector.

In some embodiments, the EEGNet module is a convolutional neural network (CNN).

The abstract spatio-temporal feature vector is a 1×30 feature vector containing feature information extracted from the sample EEG signals at different frequencies and spatial locations.

The CNN-LSTM module takes the time-frequency feature set as input, further extracts features, and obtains a dynamic time-frequency feature vector.

The dynamic time-frequency feature vector is a 1×30 feature vector containing temporal and spatial feature information from the time-frequency feature set after processing by convolutional neural networks and LSTM layers. For details on the time-frequency feature set, refer to the relevant description above.

The temporal feature module takes the sample entropy feature vector as input and normalizes it to obtain a normalized signal complexity feature vector. For more details on the sample entropy feature vector, refer to the relevant description above.

The normalized signal complexity feature vector is a standardized 1×30 feature vector containing time series complexity information of EEG signals from various leads at a same scale.

The same scale refers to a same numerical range and a same dimensionality. Through normalization, the time series complexity information of all lead EEG signals can be transformed to the same numerical range, eliminating dimensionality mismatches caused by differences in lead locations. The same applies to other unified scales mentioned below.

The spatial feature module takes the spatial feature vector as input and normalizes it to obtain a normalized spatial discriminative feature vector. For more details on the spatial feature vector, refer to the relevant description above.

The normalized spatial discriminative feature vector is a standardized 1×30 feature vector containing spatial distribution features of EEG signals from various leads on a unified scale.

The integration module concatenates and integrates the abstract spatio-temporal feature vector obtained by the EEGNet module, the dynamic time-frequency feature vector obtained by the CNN-LSTM module, the normalized signal complexity feature vector obtained by the temporal feature module, and the normalized spatial discriminative feature vector obtained by the spatial feature module, merging them into an integrated feature vector.

The integration module takes the integrated feature vector as input and obtains the binary classification result corresponding to the target EEG signal. In some embodiments, the integration module is a feedforward neural network.

In some embodiments, the temporal feature module and spatial feature module may be a segment of program/instructions in the processor/memory.

In some embodiments of the present disclosure, by normalizing the numerical ranges of sample entropy and spatial features, features of different ranges and scales can be mapped to the same interval. This avoids certain features having excessive influence on model training due to larger numerical ranges, making model training more stable and efficient, preventing model training bias, enhancing the collaborative discriminative ability of the four types of feature vectors, improving the convergence speed and accuracy of the model, and significantly increasing the recognition accuracy of fuzzy semantic targets.

In some embodiments, the abstract spatio-temporal feature vector is a row vector containing 30 first elements, wherein the first elements include frequency features and spatial location features extracted from the sample EEG signals; the dynamic time-frequency feature vector is a row vector containing 30 second elements, wherein the second elements include temporal features and spatial features extracted from the time-frequency feature set; the normalized signal complexity feature vector is a row vector containing 30 third elements, wherein the third elements include time series complexity information of each lead EEG signal at a same scale; and the normalized spatial discriminative feature vector is a row vector containing 30 fourth elements, wherein the fourth elements include spatial distribution features of each lead EEG signal at the same scale.

The frequency features refer to energy distribution features of different frequency bands (e.g., θ, α, β, γ bands) in the sample EEG signals.

The spatial location features refer to distribution patterns of EEG activity at different scalp locations and the interrelationships between different brain areas, specifically the voltage distribution features of sample EEG signals across different leads.

The spatial features refer to combined patterns formed by signal energy across different frequencies at a specific time point or within a short time window. The short time window may be preset manually.

The temporal features refer to trend features of how the energy of a specific frequency evolves over time in the signal time-frequency plot.

The time series complexity information refers to nonlinear dynamic complexity features of sample EEG signals over time as measured by sample entropy, characterizing the complexity of the sample EEG signals in the temporal dimension.

The spatial distribution features refer to task-discriminative spatial energy features optimized by spatial filters.

In some embodiments of the present disclosure, normalization processing eliminates scale differences between sample entropy and spatial features, significantly enhancing feature discriminability. The scale consistency optimization of the four types of 30-dimensional feature vectors (spatio-temporal/time-frequency/complexity/spatial) effectively strengthens the classification accuracy of fuzzy semantic targets.

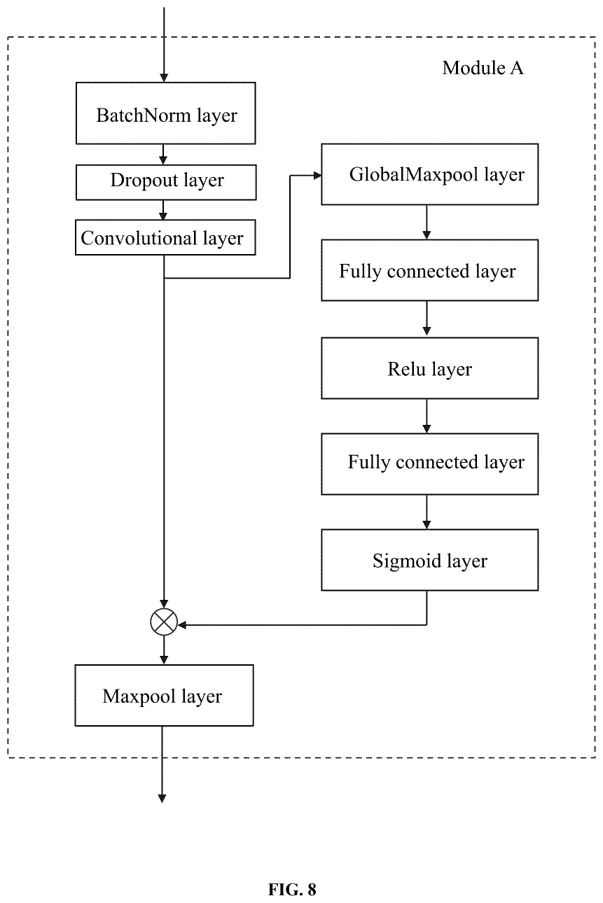

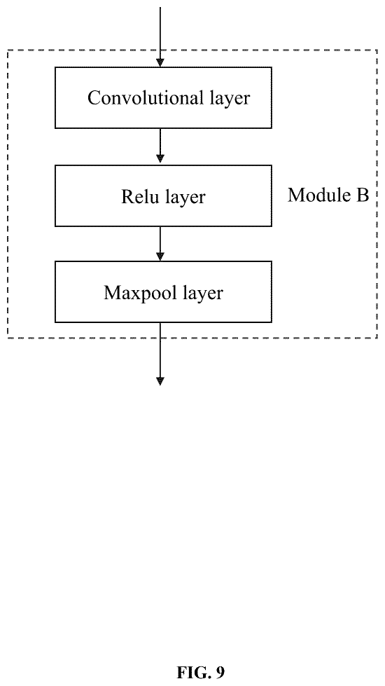

FIG. 7 is a schematic diagram illustrating an exemplary structure of an EEG classification model according to some embodiments of the present disclosure; FIG. 8 is a schematic diagram illustrating an exemplary Module A in an EEG classification model according to some embodiments of the present disclosure; and FIG. 9 is a schematic diagram illustrating an exemplary Module B in an EEG classification model according to some embodiments of the present disclosure.

In some embodiments, as shown in FIGS. 7-9, the EEGNet module includes a Batchnorm layer, two Modules A, a Dropout layer, a Fully connected layer, and a Flatten layer in turn, each of the two Modules A includes a BatchNorm layer, a Dropout layer, a convolutional layer, a GlobalMaxpool layer, a Fully connected layer, a Relu layer, a Fully connected layer, a Sigmoid layer, and a Maxpool layer in turn; the CNN-LSTM module includes a BatchNorm layer, three Modules B, an LSTM layer, a Fully connected layer, and a Flatten layer; each of the three Modules B includes a convolutional layer, a Relu layer, and a Maxpool layer in turn; the temporal feature module and the spatial feature module include a BatchNorm layer and a Flatten layer in turn, respectively; and the integration module includes a Fully connected layer, a Softmax layer, and a binary classification layer in turn.

In some embodiments, the above-mentioned layers and modules may be a program or instruction in a processor or memory.

In some embodiments of the present disclosure, the integration module combines feature vectors output by multiple modules along with the sample entropy feature vector and spatial feature vector, enabling the fusion of features extracted from different perspectives and levels. This allows the model to comprehensively integrate multi-faceted information, more holistically describe the relationship between EEG signals and fuzzy semantic targets, and enhance the model's recognition capability. The fully connected layer learns and fuses the integrated feature vectors, automatically capturing inter-feature relationships and weights to uncover latent patterns among features. The softmax activation layer then processes the output of the fully connected layer into a probability distribution format for binary classification tasks, generating probabilities for the presence and absence of fuzzy semantic targets, thereby producing the final recognition result.

In addition, certain features, structures, or characteristics of one or more embodiments of the present disclosure may be suitably combined.

Some embodiments use numbers to describe the number of components, attributes, and it should be understood that such numbers used in the description of the embodiments are modified in some examples by the modifiers “about”, “approximately”, or “substantially”. Unless otherwise noted, the terms “about”, “approximately”, or “substantially” indicate that a ±20% variation in the stated number is allowed. Correspondingly, in some embodiments, the numerical parameters used in the present disclosure and claims are approximations, which can change depending on the desired characteristics of individual embodiments. In some embodiments, the numerical parameters should consider the specified number of valid digits and employ general place-keeping. While the numerical domains and parameters used to confirm the breadth of their ranges in some embodiments of the present disclosure are approximations, in specific embodiments, such values are set to be as precise as possible within a feasible range.

For each patent, patent application, patent application disclosure, and other material cited in the present disclosure, such as articles, books, manuals, publications, documents, etc., the entire contents of which are hereby incorporated by reference herein. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions, and/or use of terms in the present disclosure shall prevail.

What is claimed is:

1. A target recognition method based on an electroencephalogram (EEG) signal in a natural reading environment executed by a processor, comprising:
presenting a fuzzy semantic target to a subject through a user interaction window on a display device;
acquiring EEG signals of the subject via a wireless EEG acquisition device to obtain a target EEG signal corresponding to the fuzzy semantic target;
determining a binary classification result corresponding to the fuzzy semantic target based on the target EEG signal through a trained EEG classification model;
in response to the binary classification result indicating that the fuzzy semantic target is recognized, determining a semantic category to which the fuzzy semantic target belongs; and
controlling, based on the semantic category, the display device to highlight annotations on the user interaction window; wherein highlighting annotations includes highlighting a text or an image related to the fuzzy semantic target and displaying associated information of the semantic category in an auxiliary area of the user interaction window.

2. The target recognition method of claim 1, comprising:
presenting a recognition target set to the subject through the user interaction window, wherein the recognition target set includes a plurality of fuzzy semantic targets;
during presenting:
obtaining a target EEG signal of the subject corresponding to each of the plurality of fuzzy semantic targets;
obtaining a binary classification result of each of the plurality of fuzzy semantic targets based on the target EEG signal of the subject corresponding to each of the plurality of fuzzy semantic targets using the trained EEG classification model;
generating a binary classification result stream based on binary classification results of the plurality of fuzzy semantic targets;
in response to the binary classification result stream meeting a negative feedback condition, generating a negative feedback instruction, wherein the negative feedback instruction is configured to control a wearable wristband to produce a vibration or a microcurrent, with a vibration intensity or a microcurrent intensity being positively correlated to an unrecognized count in the binary classification result stream; and
in response to the binary classification result stream meeting a positive feedback condition, generating a positive feedback instruction, wherein the positive feedback instruction is configured to control a head-mounted playback device to play a positive prompt tone.

3. The target recognition method of claim 1, comprising:
presenting a to-be-executed target set to the subject through the user interaction window and obtaining an EEG signal of the subject corresponding to each to-be-executed target;
determining a binary classification result of the subject for each to-be-executed target based on the EEG signal of the subject corresponding to each to-be-executed target through the trained EEG classification model; and
in response to the binary classification result of the subject for each to-be-executed target indicating that the to-be-executed target is recognized, generating an execution instruction, wherein the execution instruction is configured to drive a rehabilitation robotic arm to execute the to-be-executed target with a preset trajectory and a preset posture.

4. The target recognition method of claim 1, wherein the trained EEG classification model is obtained through training based on a sample EEG database, and the target recognition method further comprises:
performing an EEG experiment on the subject using a fuzzy semantic target recognition paradigm to obtain sample EEG signals of the subject;
constructing the sample EEG database based on the sample EEG signals and corresponding labels;
preprocessing the sample EEG signals in the sample EEG database to obtain preprocessed EEG signals; and
training an EEG classification model based on the preprocessed EEG signals to obtain the trained EEG classification model.

5. The target recognition method of claim 4, wherein the performing an EEG experiment on the subject using a fuzzy semantic target recognition paradigm to obtain sample EEG signals of the subject includes:
for each block in the fuzzy semantic target recognition paradigm:
presenting stimulus materials corresponding to the block to the subject in a predetermined sequence based on the fuzzy semantic object recognition paradigm, and controlling the wireless EEG acquisition device to acquire the sample EEG signals of the subject based on acquisition parameters; wherein the acquisition parameters include acquisition time periods and an acquisition frequency; wherein:
the controlling the wireless EEG acquisition device to acquire the sample EEG signals of the subject based on acquisition parameters includes:
controlling the wireless EEG acquisition device to perform a plurality of acquisitions on the subject at the acquisition frequency during different acquisition time periods;
recording a sample EEG signal and a corresponding label when presenting each stimulus material and a sample EEG signal and a corresponding label during a period of blank frame following each stimulus material;
after presentation of the stimulus materials for the block is completed, adjusting the acquisition parameters based on a quality of the sample EEG signals and a preset quality threshold; and
controlling the wireless EEG acquisition device to reacquire the sample EEG signals of the subject corresponding to the stimulus materials of the block based on adjusted acquisition parameters.

6. The target recognition method of claim 5, further comprising:
assessing the quality of the sample EEG signals based on a user-type feature of the subject, an acquisition time feature of the sample EEG signals, and the sample EEG signals.

7. The target recognition method of claim 6, wherein the assessing the quality of the sample EEG signals based on a user-type feature of the subject, an acquisition time feature of the sample EEG signals, and the sample EEG signals includes:
assessing the quality of the sample EEG signals based on the user-type feature, the acquisition time feature, and the EEG signals using a quality assessment model, wherein the quality assessment model is a machine learning model.

8. The target recognition method of claim 7, wherein an input of the quality assessment model further includes the stimulus materials corresponding to the sample EEG signals.

9. The target recognition method of claim 7, wherein an input of the quality assessment model further includes an ambient light intensity feature and a material carrier feature.

10. The target recognition method of claim 5, wherein the acquisition parameters further include an ambient light intensity feature and a stimulus material carrier feature, and the controlling the wireless EEG acquisition device to reacquire the sample EEG signals of the subject corresponding to the stimulus materials of the block based on adjusted acquisition parameters includes:
controlling the wireless EEG acquisition device to perform a plurality of acquisitions on the subject at the acquisition frequency during the different acquisition periods, under different ambient light intensity features, and under different material carrier features.

11. The target recognition method of claim 5, further comprising:
generating a plurality of candidate acquisition parameters and determining a rejection probability of each of the plurality of candidate acquisition parameters using a parameter determination model, wherein the parameter determination model is a machine learning model; and
determining the acquisition parameters based on the rejection probability.

12. The target recognition method of claim 4, wherein the constructing the sample EEG database based on the sample EEG signals and corresponding labels includes:
calculating a power spectrum of each lead signal;
labeling a lead signal with a value greater than twice a standard deviation of an average power spectral energy as a bad lead signal and supplementing using neighborhood interpolation;
calculating, in a single trial, a median of a variance of lead signals, and a median of a difference between each lead signal and an average of the lead signals;
labeling a trial in which either of the two medians is greater than twice a standard deviation and rejecting a sample EEG signal corresponding to the trial; and
corresponding retained sample EEG signals with labels corresponding to the retained sample EEG signals, and constructing the sample EEG database.

13. The target recognition method of claim 4, wherein the preprocessing includes filtering, downsampling, and denoising.

14. The target recognition method of claim 13, wherein the preprocessing further includes removing bioelectrical artifacts and residual noise from a downsampled EEG signal, wherein different acquisition parameters correspond to different discrimination thresholds, and the discrimination thresholds are related to a count of rejections of the sample EEG signals.

15. The target recognition method of claim 13, wherein the preprocessing further includes:
obtaining a re-referenced EEG signal by re-referencing the sample EEG signals using an average of all lead signals as a reference datum;
obtaining a filtered EEG signal by removing, from the re-referenced EEG signal, noise below 0.5 Hz and above 80 Hz and power-line interference at 50 Hz using a band-pass filter and a notch filter;
obtaining a downsampled EEG signal by downsampling the filtered EEG signal according to a sampling theorem; and
decomposing the downsampled EEG signal into a plurality of independent components using independent component analysis (ICA), calculating a frequency feature of each component; and removing bioelectrical artifacts and residual noise.

16. The target recognition method of claim 15, further comprising:
obtaining a signal time-frequency plot of each lead by performing time-frequency analysis on the preprocessed EEG signals using continuous wavelet transform (CWT);
obtaining a time-frequency feature set of a preprocessed EEG signal of each lead by extracting an image feature of the signal time-frequency plot using a convolutional neural network;
obtaining a sample entropy feature vector of each trial by calculating a sample entropy feature of the preprocessed EEG signal of each lead in the trial; and
obtaining a spatial feature vector of each trial by extracting a spatial feature of the preprocessed EEG signal of each lead in the trial using a common spatial pattern.

17. The target recognition method of claim 1, wherein the trained EEG classification model is obtained through training based on a sample EEG database, and the target recognition method further comprises:
performing an EEG experiment on the subject using a fuzzy semantic target recognition paradigm to obtain sample EEG signals of the subject;
constructing the sample EEG database based on the sample EEG signals and corresponding labels;
randomly dividing the sample EEG signals in the sample EEG database into a training set and a test set, using the sample EEG signals as an input and the corresponding labels as a target output;
training an EEG classification model using the sample EEG signals and the corresponding labels in the training set to obtain the trained EEG classification model which outputs the binary classification result; and
applying the test set to the trained EEG classification model, and analyzing performance and robustness of the trained EEG classification model to explore an association between an EEG activity and a fuzzy semantic target recognition activity.

18. The target recognition method of claim 1, wherein the trained EEG classification model includes an EEGNet module, a CNN-LSTM module, a temporal feature module, a spatial feature module, and an integration module; and the determining a binary classification result corresponding to the fuzzy semantic target based on the target EEG signal through a trained EEG classification model includes:
determining an abstract spatio-temporal feature vector based on preprocessed EEG signals using the EEGNet module, wherein the EEGNet module is a convolutional neural network;
determining a dynamic time-frequency feature vector based on a time-frequency feature set using the CNN-LSTM module;
determining a normalized signal complexity feature vector based on a sample entropy feature vector using the temporal feature module;
determining a normalized spatial discriminative feature vector based on a spatial feature vector using the spatial feature module;
concatenating the abstract spatio-temporal feature vector, the dynamic time-frequency feature vector, the normalized signal complexity feature vector, and the normalized spatial discriminative feature vector using the integration module to obtain an integrated feature vector; and
obtaining the binary classification result corresponding to the target EEG signal based on the integrated feature vector using the integration module, wherein the integration module is a feedforward neural network.

19. The target recognition method of claim 18, wherein:
the abstract spatio-temporal feature vector is a row vector containing 30 first elements, wherein the first elements include frequency features and spatial location features extracted from the sample EEG signals;
the dynamic time-frequency feature vector is a row vector containing 30 second elements, wherein the second elements include temporal features and spatial features extracted from the time-frequency feature set;
the normalized signal complexity feature vector is a row vector containing 30 third elements, wherein the third elements include time series complexity information of each lead EEG signal at a same scale; and
the normalized spatial discriminative feature vector is a row vector containing 30 fourth elements, wherein the fourth elements include spatial distribution features of each lead EEG signal at the same scale.

20. The target recognition method of claim 18, wherein the EEGNet module includes a Batchnorm layer, two Modules A, a Dropout layer, a Fully connected layer, and a Flatten layer in turn, each of the two Modules A includes a BatchNorm layer, a Dropout layer, a convolutional layer, a GlobalMaxpool layer, a Fully connected layer, a Relu layer, a Fully connected layer, a Sigmoid layer, and a Maxpool layer in turn;
the CNN-LSTM module includes a BatchNorm layer, three Modules B, an LSTM layer, a Fully connected layer, and a Flatten layer; each of the three Modules B includes a convolutional layer, a Relu layer, and a Maxpool layer in turn;
the temporal feature module and the spatial feature module include a BatchNorm layer and a Flatten layer in turn, respectively; and
the integration module includes a Fully connected layer, a Softmax layer, and a binary classification layer in turn.
